(19) United States
(12) Patent Application Publication
(10) Pub. No.: US 2022/0163529 A1
(43) Pub. Date: May 26, 2022

(54) MONOCLONAL ANTIBODY FOR SARS-CoV-2 SPIKE PROTEIN

(71) Applicants: Arizona Board of Regents on behalf of Arizona State University, Scottsdale, AZ (US); Halberd Corporation, Jackson Center, PA (US)

(72) Inventors: Qiang Chen, Chandler, AZ (US); Mitchell Steven Felder, Hermitage, PA (US)

(21) Appl. No.: 17/528,891

(22) Filed: Nov. 17, 2021

Related U.S. Application Data
(60) Provisional application No. 63/116,561, filed on Nov. 20, 2020.

(51) Int. Cl. G01N 33/569; G01N 15/14

(52) U.S. Cl. CPC G01N 33/56983; G01N 15/1434; G01N 2015/1006; G01N 2333/165; G01N 2469/10

(57) ABSTRACT

An embodiment provides a method for detection of the COVID-19 virus, including: obtaining a body fluid from a patient; exposing the body fluid to at least one binding antibody, wherein the at least one binding antibody binds to an antigen in the body fluid; forming an antigen antibody complex; and determining the presence of the antigen antibody complex. Other aspects are described and claimed.

### CROSS REFERENCE TO RELATED APPLICATION

This application claims priority to U.S. Provisional Patent Application Ser. No. 63/116,561, filed on Nov. 20, 2020, and entitled “MONOCLONAL ANTIBODY FOR SARS-CoV-2 SPIKE PROTEIN,” the contents of which are incorporated by reference herein.

### FIELD

This application relates generally to detection of SARS-CoV-2, and, more particularly, to the spike protein of SARS-CoV-2.

### BACKGROUND

Coronaviruses represent a group of viruses that may lead to respiratory tract infections. These infections may range from mild to lethal. Coronaviruses may cause severe acute respiratory syndrome (SARS) and Middle East respiratory syndrome (MERS). A novel coronavirus (COVID-19) has led to a global pandemic causing a public health and economic crisis. Severe acute respiratory syndrome coronavirus 2 (SARS-CoV-2) is the strain of coronavirus that causes coronavirus disease 2019. Transmission may be through close contact of individuals and via respiratory droplets such as coughs or sneezes. Faster and more accurate methods of identifying and treating individuals infected with COVID-19 could mitigate the global pandemic.

### BRIEF SUMMARY

In summary, one embodiment provides a method for detection of the COVID-19 virus, comprising: obtaining a body fluid from a patient; exposing the body fluid to at least one binding antibody, wherein the at least one binding antibody binds to an antigen in the body fluid; forming an antigen antibody complex; and determining the presence of the antigen antibody complex.

Another embodiment provides a method for detection of the COVID-19 virus, comprising: obtaining a body fluid from a patient; exposing the body fluid to at least one binding antibody, wherein the at least one binding antibody binds to the SARS-CoV-2 spike (S) protein; forming a virion antibody complex; and determining the presence of the virion binding antibody complex.

A further embodiment provides a method for detection of the COVID-19 virus, comprising: obtaining a body fluid from a patient; exposing the body fluid to at least one binding antibody on a microarray, wherein the at least one binding antibody binds to the SARS-CoV-2 spike (S) protein, wherein the at least one binding antibody comprises a fluorescent tag; and detecting the presence of the SARS-CoV-2 spike (S) protein using a light source to detect the fluorescent tag.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates a flow diagram of an example method for detection of the spike protein of SARS-CoV-2 using a monoclonal antibody.

FIG. 2 illustrates an example binding curve of a monoclonal antibody for the spike protein of SARS-CoV-2.

FIG. 3A illustrates example flow cytometry data for monoclonal antibodies for the spike protein of SARS-CoV-2 stained with anti-S mAb1.

FIG. 3B illustrates example flow cytometry data for monoclonal antibodies for the spike protein of SARS-CoV-2 stained with anti-S mAb1 that have been fluorescently labeled with Alexa488.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

COVID-19 has spread worldwide and become a global pandemic. The loss of life, suffering, and economic struggles have reached all corners of the globe. Symptoms may manifest about 2-14 days after exposure. The symptoms may include fever, chills, cough, shortness of breath, difficulty breathing, fatigue, muscle/body aches, new loss of taste/smell, sore throat, congestion, runny nose, nausea, vomiting, or diarrhea. More severe symptoms may include trouble breathing, persistent pain/pressure in the chest, confusion, inability to wake or stay awake, or bluish lips/face. Some cases may require hospitalization and even intensive care unit healthcare. Because of the novelty of the virus, very few tests exist that are specific for COVID-19. What is needed is a rapid and accurate identification of COVID-19 in a patient.

Accordingly, an embodiment provides a method for determining the presence of COVID-19 in body fluid of a patient. The identification may be rapid in time. The identification may be from a patient's body fluid. The body fluid may be blood, CSF (cerebrospinal fluid), mucus, saliva, or any bodily fluid. The body fluid may be from a patient which may contain COVID-19 virions. In an embodiment, the body fluid may be exposed to at least one binding antibody. In an embodiment, a treatment may be applied to the body fluid. In an embodiment, a treatment may comprise exposing the body fluid to a binding antibody. The binding is to an antigen specific to the spike protein of SARS-CoV-2. In an embodiment, the method may determine the presence or absence of the virion binding antibody complex. The binding antibody may include, for example, a fluorescent antibody, a luminous antibody, combinations thereof, or the like. The method may utilize different techniques for determining the presence or absence of the virion binding antibody complex as described below.

The illustrated example embodiments will be best understood by reference to the figures. The following description is intended only by way of example, and simply illustrates certain example embodiments.

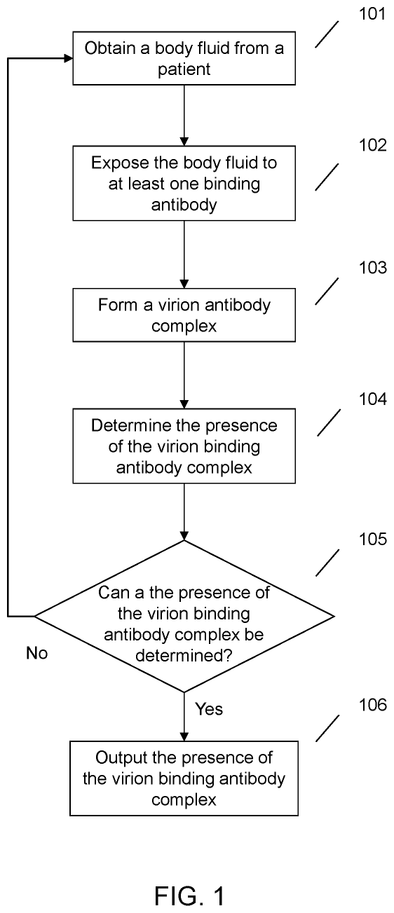

Referring to FIG. 1, an example device and method for determining the presence of COVID-19 in a body fluid. The method may use a monoclonal antibody. The antibody may be directed to the spike protein of SARS-CoV-2. The antibody may be a fluorescent tag. In an embodiment, the body fluid may be exposed to at least one binding antibody, form a virion antibody complex. The system and method may determine the presence of the virion binding antibody complex. The method and system may be used to determine if a patient has antibodies for the COVID-19 disease. It should be understood that the method and system described herein may be used for diagnostic purposes. In other words, the method may be used as a test kit for example at a medical facility, a testing facility, at home, or the like.

At 101, in an embodiment, a method may identify or detect COVID-19. The identification may be rapid in time. The identification may be from a patient's body fluid. The body fluid may be blood, CSF (cerebrospinal fluid), mucus, saliva, or any bodily fluid. The body fluid may be from a patient which may contain COVID-19 virions. A virion may be a complete ineffective form of a virus outside of a host cell. A virion may be able to bind to a monoclonal antibody selective for the virion component. The component may include, but is not limited to genetic material such as DNA or RNA, protein, or the like.

For example, a sample or a body fluid may be withdrawn from a patient using standard medical techniques. Techniques may include sterile cotton swab, blood draw, lumbar puncture, or another accepted form of body fluid collection. Collection may include methodology to preserve the sample such as temperature regulation, sterile techniques, stability agents, buffers, or the like.

At 102, in an embodiment, the body fluid may be exposed to at least one binding antibody. For example, the antibody may be a monoclonal antibody and may selectively bind the spike protein of SARS-CoV-2. At 103, in an embodiment, the COVID-19 virions present in the body fluid may form a virion antibody complex with the binding antibody.

In an embodiment, a treatment may be applied to the body fluid. In an embodiment, a treatment may comprise exposing the body fluid to a binding antibody. The binding be to an antigen specific to the spike protein of SARS-CoV-2. The specific antigen may include at least one antigen. The antigen may include the of SARS-CoV-2 spike glycoprotein. Other antigens may be included for testing as well. Other antigens may include Covid-19 M-Protein, Covid-19 Hemoglutinesterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, nsp10-nsp16 complex forming virion antibody complexes, and combinations thereof. The binding antibody and Covid-19 specific antigen form a virion antibody complex (FIL T Ab-TPA complex).

In an embodiment, the spike protein of SARS-CoV-2 antibody may contain an albumin moiety. This antibody may target and rapidly identify COVID-19 antigens. The antibody may or may not include a fluorescent tag. The fluorescent tag may be used for detection techniques. The fluorescent tag may be Alexa-488, Indocyanine green (ICG) or the like.

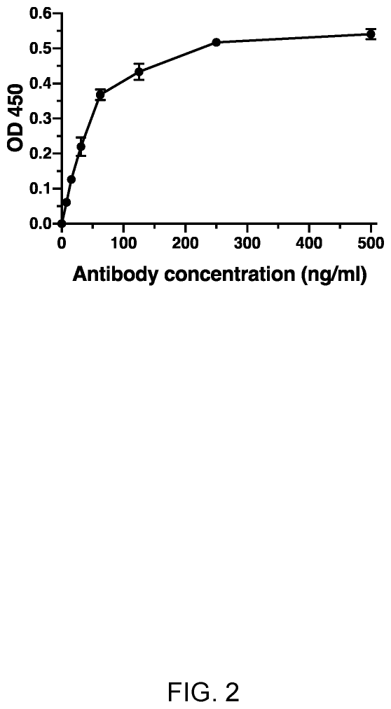

Example binding data are illustrated in FIG. 2. Referring to FIG. 2, ELISA of anti-Spike (S) protein of SARS-CoV-2 mAb (mAb1) binding to the spike protein is illustrated. Serial dilutions of mAb1 were incubated on plates coated with the spike protein and detected with a HRP-conjugated anti-human-kappa antibody. This indicates the specific binding of mAb1 to the S protein.

At 104, in an embodiment, the method may determine the presence or absence of the virion binding antibody complex. The binding antibody may include, for example, a fluorescent antibody, a luminous antibody, combinations thereof, or the like. The concentration of binding antibody may be made as high as necessary for the identification of extremely small, e.g., picogram/microliter, concentrations of the final virion binding antibody complex. The signal from the virion binding antibody complex may be amplified. The amplification may be necessary to identify a signal.

The method may utilize different techniques for determining the presence or absence of the virion binding antibody complex. As an example, a dialysis or a variant of dialysis may be used. The dialysis may be used to remove the fluorescent antibody-antigen complex. This may allow for a rapid identification of a COVID-19 sample. Such technique may be automated, controlled by a computer system, or the like. The system may use a threshold, limits, alarms, or the like.

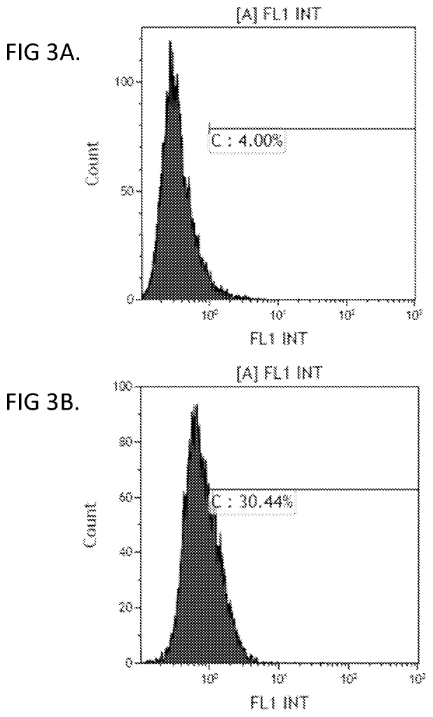

In an embodiment, the methods may use flow cytometry analysis of fluorescent labelled antibodies relating to COVID-19. See FIG. 3A and FIG. 3B. For example, flow cytometry analysis of fluorescent mAbs against SARS-CoV-2 spike (S) protein may be performed. For example, K562 cells may be fixed with 4% PFA (Paraformaldehyde) then permeabilized with 0.1% saponin in PBS (Phosphate-buffered saline). Cells may then be stained with anti-S mAb1 (FIG. 3A) or with mAb1 that has been fluorescently labeled with Alexa488 (FIG. 3B). Stained cells may be processed by flow cytometry. A rightward shift of fluorescent intensity in FIG. 3B as compared to FIG. 3A indicates the fluorescent labeling of mAb1.

In an embodiment, a method may utilize a designer fluorescent antibody with an attached macromolecular moiety. The macromolecular moiety, attached to the antibody, may be 1.000 mm to 0.00001 mm in diameter. Disclosed diameters are illustrative and may vary. The antibody-macromolecular moiety-targeted antigen complex would then be blocked for analysis, by using a series of microscreens which contain openings with a diameter 50.00000% to 99.99999% less than the diameter of the designer antibody-macromolecular moiety.

In an embodiment, methodology comprising the removal of the targeted antigen(s)/TA(s) by using a designer fluorescent antibody containing an iron (Fe) moiety. This will then create an Fe-fluorescent Antibody-Antigen (COVID-19/virion) complex. This iron containing complex may then be efficaciously removed using a strong, localized magnetic force field, which may be identified as positive.

In an embodiment, a variant of gel filtration chromatography, which may be utilized for the rapid identification of COVID-19. The fluorescent antibody-target antigen would be used to transport the sample through a size exclusion column that would be used to separate the fluorescent antibody-target antigen by size and molecular weight.

In an embodiment, a methodology using a molecular weight cutoff filtration may be employed. Molecular weight cut-off filtration refers to the molecular weight at which at least or approximately 80% of the target antigen(s)/TA(s) may be prohibited from membrane diffusion.

In an embodiment, a removal methodology for the fluorescent antibody-target antigen(s) may be used. The removal methodology may be selected from a group comprising a mechanical filter, a chemical filter, a dialysis machine, a molecular filter, molecular adsorbent recirculating system (MARS), a plasmapheresis unit, or combinations thereof.

For example, virions may be captured using antibody microarrays. The microarray may contain one or more binding antibody. In an embodiment, the binding antibody may comprise a fluorescent antibody (FI). In another embodiment, the binding antibody may comprise a luminescent (Lu) antibody. The microarray may comprise a plurality of antibodies fixed on a solid surface. The solid surface may be any suitable material. The microarray material may be transparent, such as glass, plastic, silicon, combinations thereof, or the like. The microarray may allow detection of at least one virion antibody complex. The microarray can comprise a plurality of monoclonal antibodies attached at high density on the solid surface. Typically, the microarray may contain millions of antibodies. Exposure of the virion to the binding antibodies on the microarray creates the virion. The complex may be tracked using an appropriate sensor. To identify the virion antibody complex after exposure in the microarrays, the body fluid may then be forced through a container preferably constructed from a transparent material, which exposes the virion antibody complex to a light-sensing device. The sensing device may also create an enlarged, magnified visual image of virion antibody complex. A concentrated and focused intense energy beam, such as light, is then used to properly illuminate the virion antibody complex within the body fluid. Each virion antibody complex may be rapidly identified. The virion antibody complex may also be identified and tracked using optical or digital enhancement or magnification.

At 105, in an embodiment, if a virion antibody complex cannot be determined, the system may continue to determine the presence of another virion antibody complex in the body fluid. Alternatively, the method or system may determine that the patient body fluid does not contain the virion. For example, the patients does not have COVID-19. Additionally or alternatively, the system may output an alarm, log an event, or the like. If a filter life can be determined, the system may provide an output at 106. The virion binding antibody complex determination may be an output that is provided to a device in the form of a display, printing, storage, audio, haptic feedback, or the like. Alternatively or additionally, the output may be sent to another device through wired, wireless, fiber optic, Bluetooth®, near field communication, or the like

The various embodiments described herein thus represent a technical improvement to antibodies directed to the spike protein of SARS-CoV-2. Using the techniques as described herein, an embodiment may use a method to determine the presence or absence of SARS-CoV-2 in a sample from a patient.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a hand held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for detection of the COVID-19 virus, comprising:
obtaining a body fluid from a patient;
exposing the body fluid to at least one binding antibody, wherein the at least one binding antibody binds to an antigen in the body fluid;
forming an antigen antibody complex; and
determining the presence of the antigen antibody complex.

2. The method of claim 1, wherein the body fluid is selected from the group consisting of: blood, CSF, mucus, and saliva.

3. The method of claim 1, wherein the at least one binding antibody comprises a fluorescent tag.

4. The method of claim 3, wherein the fluorescent tag comprises Alexa-488.

5. The method of claim 1, wherein the determining comprises identifying the fluorescent tag using flow cytometry.

6. The method of claim 1, wherein the at least one binding antibody comprises a luminous tag.

7. The method of claim 1, wherein the antigen comprises a SARS-CoV-2 spike (S) protein.

8. The method of claim 1, further comprising removing the antigen antibody complex from the body fluid.

9. The method of claim 8, wherein the removing is performed by a size exclusion method.

10. The method of claim 1, wherein the antigen is selected from the group consisting of: Covid-19 M-Protein, Covid-19 Hemoglutinesterase dimer, Covid-19 Envelope, Covid-19 E-Protein, Covid-19 N-Protein, nsp (non-structural protein) 12 RNA-dependent RNA polymerase (nsp 12), nsp (non-structural protein) 7, nsp 8, nsp 14, nsp 12-nsp 7-nsp 8 complex, nsp7-nsp8 complex, nsp10-nsp14 complex, and nsp10-nsp16 complex.

11. A method for detection of the COVID-19 virus, comprising:
obtaining a body fluid from a patient;
exposing the body fluid to at least one binding antibody, wherein the at least one binding antibody binds to the SARS-CoV-2 spike (S) protein;
forming a virion antibody complex; and
determining the presence of the virion binding antibody complex.

12. The method of claim 11, wherein the body fluid is selected from the group consisting of: blood, CSF, mucus, and saliva.

13. The method of claim 11, wherein the at least one binding antibody comprises a fluorescent tag.

14. The method of claim 13, wherein the fluorescent tag comprises Alexa-488.

15. The method of claim 11, wherein the determining comprises identifying the fluorescent tag using flow cytometry.

16. The method of claim 11, wherein the at least one binding antibody comprises a luminous tag.

17. The method of claim 11, wherein the at least one binding antibody for SARS-CoV-2 spike (S) protein is bound to a microarray of a transparent material allowing exposure of the virion binding antibody complex to a light source.

18. The method of claim 11, further comprising removing the antigen antibody complex from the body fluid.

19. The method of claim 18, wherein the removing is performed by a size exclusion method.

20. A method for detection of the COVID-19 virus, comprising:
obtaining a body fluid from a patient;
exposing the body fluid to at least one binding antibody on a microarray, wherein the at least one binding antibody binds to the SARS-CoV-2 spike (S) protein, wherein the at least one binding antibody comprises a fluorescent tag; and
detecting the presence of the SARS-CoV-2 spike (S) protein using a light source to detect the fluorescent tag.
